(19) United States
(12) Patent Application Publication
(10) Pub. No.: US 2025/0049377 A1
(43) Pub. Date: Feb. 13, 2025

(54) METHOD OF NON-INTRUSIVELY MEASURING BRAINWAVE ACTIVITY IN A FETUS

(71) Applicants: Allon Guez, Penn Valley, PA (US); Offer Erez, Doar Na Negev (IL); Helen Guez, Penn Valley, PA (US)

(72) Inventors: Allon Guez, Penn Valley, PA (US); Offer Erez, Doar Na Negev (IL); Helen Guez, Penn Valley, PA (US)

(21) Appl. No.: 18/786,981

(22) Filed: Jul. 29, 2024

Related U.S. Application Data
(60) Provisional application No. 63/531,027, filed on Aug. 7, 2023.

(51) Int. Cl. A61B 5/369; A61B 5/00; A61B 5/31

(52) U.S. Cl. CPC A61B 5/369; A61B 5/31; A61B 5/7275; A61B 2503/02; A61B 2503/045

(57) ABSTRACT

A method of non-intrusively measuring brainwave activity in a fetus is provided. The method provides the steps of: directing at least a first monitoring sensor to a pregnant woman's body; measuring a first set of signals received by the at least first monitoring sensor; filtering non-fetal brainwave signals from the first set of signals, resulting in fetal brainwave activity signals; and determining fetal brainwave activity from the fetal brainwave activity signals.

### CROSS-REFERENCE TO RELATED APPLICATION

The present application claims the benefit of U.S. Provisional Patent Application Ser. No. 63/531,027, filed on Aug. 7, 2023, which is incorporated herein by reference in its entirety.

### BACKGROUND OF THE INVENTION

### Field of the Invention

The invention relates to a method of non-intrusively measuring brainwave activity in a fetus.

### Description of the Related Art

Fetal brain activity and function remain enigmatic despite the significance of this information to management of pregnancy as chronic (fetal growth restriction-FOR), and acute (hypoxic ischemic encephalopathy-HIE) events affecting this process, are implicated in the pathogenesis of childhood neurodevelopmental delays and cerebral palsy (CP), carrying a tremendous personal and social price.

It would be beneficial to be able to non-intrusively measure brainwave activity in a fetus and to identify the source of neurological issues in a child based on the fetal brainwave measurements.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a method of non-intrusively measuring brainwave activity in a fetus. The method comprises the steps of: (a) directing at least a first monitoring sensor to a pregnant woman's body; (b) measuring a first set of signals received by the at least first monitoring sensor; (c) filtering non-fetal brainwave signals from the first set of signals, resulting in fetal brainwave activity signals; and (d) determining fetal brainwave activity from the fetal brainwave activity signals.

In another embodiment, the invention is a method of non-intrusively measuring brainwave activity in a fetus, the method comprising the steps of: (a) receiving data from a first set of sensors; (b) filtering non-fetal brainwave signals from the data, resulting in fetal brainwave activity signals; (c) determining fetal brainwave activity from the fetal brainwave activity signals; (d) repeating steps (a)-(c) a plurality of times; and (e) determining a trend of fetal functional brain development and well-being based on the fetal brainwave activity.

In still another embodiment, the present invention provides a method of non-intrusively measuring brainwave activity in a fetus, the method comprising the steps of: (a) directing at least a first monitoring sensor to a pregnant woman's body; (b) measuring a first set of signals received by the at least first monitoring sensor; (c) repeating steps (a) and (b) a plurality of times; and (d) using an independent method to identify fetal brainwave signals in the first set of signals.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a timeline of brain development from conception to adulthood;

FIG. 2 is a graph comparing normal fetal and neonatal fetal EEGs;

FIG. 3 is a block diagram of a wearable non-invasive EEG device according to an exemplary embodiment of the present invention;

FIG. 4 is a block diagram of an amplifier used with the device of FIG. 3;

FIG. 5 is a schematic drawing showing the comparison of a fetal EEG with a neonate EEG from the same neonate;

FIG. 6 is a schematic drawings showing an exemplary progression of EEG recordings throughout gestation;

FIG. 7 is a schematic drawing showing biomarkers specific features of a neonatal EEG are examined in a term fetus;

FIG. 8 is a schematic drawing showing a magnetoenchephalograph (MEG) machine, with data obtained from the MEG compared to data obtained from a fetal EEG;

FIG. 9 is a schematic of the use of Blind Source Separation according to an exemplary embodiment of the invention;

FIG. 10 is a schematic of the use of Blind Source Separation according to an alternative exemplary embodiment of the invention;

FIG. 11 is an exemplary placement of sensors on a pregnant woman's abdomen; and

FIG. 12 is a schematic drawing of artificial intelligence/machine learning method predicting fetal brain activity.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

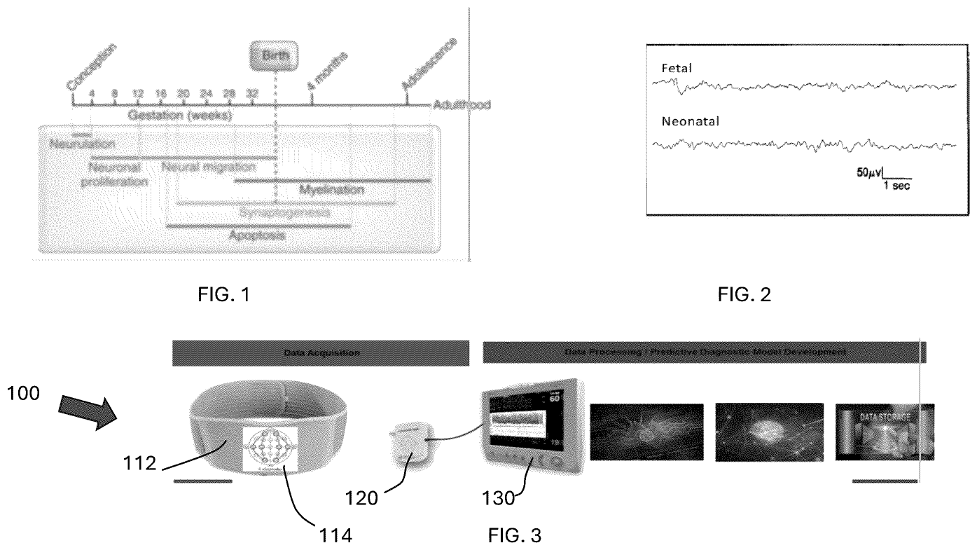

The structural and functional development of the fetal brain is a continuous process from conception to early adulthood. See FIG. 1. The fetal brain is among the earliest organs to develop. The major development occurs during the second half of pregnancy. Abnormal functional brain development has dire consequences later in life. Nevertheless, despite its importance, functional assessment of fetal brain during gestation is not part of routine prenatal care. The current practice of prenatal follow up on fetal brain development includes a sonographic assessment of fetal brain anatomy. In recent years, Magnetic Resonance Imaging (MRI) and Magnetoencephalography (MEG) have been used to study functional fetal brain development. However, these technologies are expensive, non-flexible and require special hardware and training that preclude them from being implemented in routine prenatal care. The direct non-invasive measurement of fetal brain activity is technically challenging due to the low amplitude of the fetal brain electric activity signals in comparison to background noise resulting from, fetal and maternal cardiac activity, uterine and maternal muscle electrical activity, and other environmental interference. If the function of the brain itself is to be monitored, electroencephalogram (EEG) is a direct method to measure the electrical signals produced by the brain.

EEG is a tool of functional brain assessment, and the utility of electrophysiological techniques in characterizing the maturity of neuronal activity in infants and young children is well established. Indeed, EEG studies have shown that the shape and latency of acoustic evoked potentials (AEPs) change rapidly during the first months and years of life. Similar changes were observed following visual and somatosensory stimulations.

Table 1 shows classification of EEG signal frequency band 2021 22. EEG has become a part of a standard of care in neonatal functional brain assessment in premature infants, and neonates with hypoxic ischemic encephalopathy.

TABLE 1
Classification of EEG Signal Frequency Bands

| | |
|---|---|
| Delta | 1-3 Hz |
| Theta | 3-6 Hz |
| Alpha | 6-9 Hz |
| Beta | 12-30 Hz |
| Gamma | 30-50 Hz |

Previous reports suggested that prenatal and neonatal EEG are similar in their amplitude and patterns (FIG. 2). Fetal EEG was recorded as early as 45 days after conception (8.5 weeks of gestation), suggesting that fetal brain function can be detected already at early stages of gestation. Studies demonstrated that fetal EEG can be recorded abdominally during pregnancy and transvaginal (during labor)

However, the success rate of fetal EEG acquisition even during labor when the electrode was in direct contact with the fetal's head was about 50%. Transient changes in fetal EEG during labor were observed in association with maternal meperidine administration, forceps deliveries, variable and late fetal heart rate decelerations. Non-transient fetal changes were reported in association with abnormal infant neurologic examination. Indeed, it has been reported that out of 23 fetuses with sharp waves EEG recording during labor 10 (43.5%) had abnormal neurological disorders at the age of one year vs. only 10 out of 73 (13.7%) fetuses with a normal fetal EEG recording (p=0.002).

The present invention provides a method to non-intrusively measure brainwave activity in a fetus as well as to identify the source of neurological issues in a child based on the fetal brainwave measurements. FIG. 3 is a block diagram of an exemplary wearable non-invasive EEG monitoring system 100 for fetal development according to an exemplary embodiment of the present invention. System 100 includes a wearable data acquisition component 110 with wireless transmission capability and a wireless receiver 120 that is electronically connected to a data processor/diagnostic device with display (processor) 130.

Data acquisition component 110 is mostly hardware based, while processor 130 is mostly software based on a Laptop/PC. The Laptop/PC or extended PC screens is a primary source of viewing acquired raw EEG signals while the signals are being stored on the Laptop/PC hard disk drive (HDD) for future use. Exemplary software can be original equipment manufacturer (OEM) proprietary software, if applicable, and 2) MATLAB. MATLAB's EEG Lab and artificial neural networks toolboxes are used for digital signal processing and development of the diagnostic model(s) respectively.

Data acquisition component 110 comprises a wearable band 112 similar to those used with known maternity belts. Band 112 has pockets or handles (not shown) sewn onto band 112 to house the parts of the wireless EEG amplifier 114 for ambulatory data acquisition. Spreading the electrodes along the maternal abdomen will allow signals to be captured even in cases of fetal movements and adjust for the movement during data analysis.

Data acquisition component 110 includes electrode array patches with onboard power and wireless communication, shown in FIG. 3. Two major factors are considered for selection of the electrodes, namely, ease of application and impedance. While dry electrodes can be used since they do not require long setup time that include application of gel or saline, which may cause skin irritations. However, a drawback of dry electrodes is the very high skin-electrode impedance, and maybe susceptible to motion artifact-crucial elements for EEG recordings. Moreover, wet or gel type electrodes give better sign to noise ratio (SNR).

The impedance being considered is electrode skin-electrode impedance. To reduce the effects of the impedances, active electrodes are desired. Due to the low amplitude of fetal EEG, it is desired to reduce the skin-electrode impedance which in turn will reduce noise interference from exogenous artifacts. An exemplary EEG electrode is the g.SAHARA active electrode, manufactured by g.tech medical engineering GmbH, located in Schiedlberg, Austria. Also, EMG electrodes can be obtained from g.tec as well.

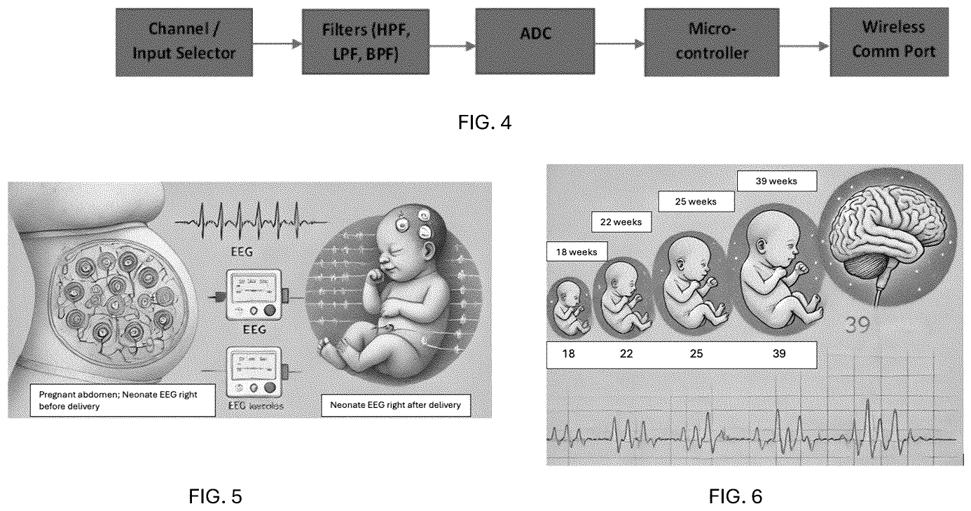

FIG. 4 is a block diagram showing the components of EEG amplifier 114. An exemplary amplifier 114 can be an 8-channel amplifier (reference, ground, and EMG not included), with gain selector, active band filter (HPF), low-band filter (LPF), and band-pass filter (BPF) included. Also included is an analog to digital converter (ADC) and microcontroller.

Based on knowledge that the term fetal EEG is that of the neonate, each fetus-neonate can be used as its own control to validate the fetal EEG recorder. The recordings are performed on pregnant women with a normal pregnancy who are about to undergo elective cesarean at term without labor. An hour prior to surgery, a fetal EEG is performed. The EEG recorder 110 is placed on the maternal abdomen with the maternity band 112 that is adjusted to the position of the fetal head (validated by ultrasound). Within eight hours after delivery, the newborn undergoes a neonatal aEEG testing and the pre and post recordings are compared, as shown in FIG. 5. This phase is used to validate the reading of the fetal EEG. The recording obtained at this stage is used also to fine-tuning and enhancement of the fetal EEG learning process and reading algorithm.

An exemplary process includes data extraction from 10 fetus (prior to cesarean section) and 10 neonates postpartum) totally 10 hours of recording in each group over a period of six (6) months. All data is preprocessed and filtered to remove artifacts, and then stored into epochs for analysis and future model training. Next, comparison of the EEG signals during prenatal stage is compared with EEG signal obtained postpartum to validate monitoring system 100. The reading of the EEG recording is to be performed by trained neonatologists. Finally, signal processing and machine learning algorithms/techniques are used for features extraction.

The normality of the EEG activity is then investigated by normal distribution attributes, skewness, kurtosis, and inverse normal plot. Since the data of the recording of the pre- and post-natal EEG recording is not independent, the McNemar test is used for dichotomous variables and paired t-test or Wilcoxon signed ranks test continuous variables.

Next, the prenatal readings of fetal EEG recording are compared to the post-natal reading of neonatal aEEG of the same fetus/neonate. The post-natal EEG signals are sampled from two parietal electrodes located at P3 and P4 areas according to the international 10/20 EEG system. Frequencies under 2 Hz and over 15 Hz are asymmetrically filtered after initial pre-amplification, then the EEG signal is rectified, smoothened, whitened, and compressed to a set logarithmic scale. The final output, reflecting the maximum and minimum amplitudes of the original EEG, is displayed at a speed of 6 cm per hour. Patterns of each ten minute segment of the aEEG recording are classified into: 1. Isoelectric; 2. Burst suppression; 3. Low discontinuous (baseline below 3 microvolts); 4. High discontinuous (baseline between 2 and 5 microvolts); 5. Continuous; and 6. Artifact. The patterns of the fetal and neonatal EEG are compared based on the classification methods described above by trained neonatologists and the similarity between the fetal and neonatal recordings will be determined. To assess the similarity scoring, Cohen's and Fleiss' Kappa tests are used for ordinal categorical variables, the weighted and Fleiss' Kappa tests for ordinal categorical data and interclass correlation and Bland-Altman plots for continues variables.

A significant challenge is disambiguating noise from true fetal neural signals. Several approaches can be used to reduce such risks and solve this significant challenge: 1) MAXIMIZING Signal to Noise Ratios-use the best and most recent EEG and EMG electrodes, skin interfaces, sampling rates, bits resolutions, preprocessing (normalization, outliers removal etc.) and processors available in the market, which allow for the best signal to noise ratio possible.
2) EXPLOITING PRIOR KNOWLEDGE: Unlike the fetal EEG, which is practically UNKNOWN, the “disturbances” to such identification (Muscle, motion, mother and fetal cardiac, digestive tract, and environmental “noise”) are rather “familiar” and have been very well studied. A reliance on available prior knowledge in characterizing each of the quite FAMILIAR disturbance sources can be used. Thus, their signal features can be identified and eliminated from the total integrated measured signal. Following the elimination of known signals, using remaining machine learning and signal estimation methods are used for identifying and characterizing the fetal EEG. This approach can be employed for all EMG, EKG, motion artifacts that are independently measured by other sensors as well as other known factors.
3) FETAL CNS NATURAL GROWTH AND DEVELOPMENT as a distinguishing factor in enhancing fetal EEG patterns recognition. Recognizing the fact that while the other disturbance sources, though nonstationary in general, do not necessarily change with the advanced pregnancy. The fetal EEG is expected to evolve and get stronger with each day of advancing pregnancy, therefore by time varying signal estimation methods that observation is exploited for improved recognition of fatal EEG after all of the estimated other disturbance sources which are assumed to not change monotonically and grow monotonically in time are subtracted.
4) DIRECT ACCESS OPPORTUNITY FOR METHOD VALIDATION: Compare the neonatal EEG that is directly measured immediately after delivery with the one that was indirectly measured during the hour prior to delivery. Finally, the proposed machine learning algorithms can be TAILORED to the signals processing strategies described above and are NOT arbitrary. The Residual signals upon subtraction of “known” disturbances are used for training and validation of fetal EEG estimation. Furthermore, training upon residuals obtained in advanced pregnancy samples (where the fetal brain is most developed and therefore fetal EEG is strongest), can generate the Machine Learning algorithms used in the EARLIER pregnancy stages for an expected more robust identification. The signal processing an EEG extraction during aim number 1) relies on the reference data of Post-delivery neonatal EEG recording that can be used through supervised learning techniques for training and extracting the correct fetal EEG. Those reference EEG will be used to extract relevant parameters for more conventional signal processing and filtering algorithms that includes bandwidth magnitude and other features of the fetal EEG.

Various signals processing and machine learning methods are considered for the task of accurate fetus EEG extraction from the acquired data. Prior knowledge regarding fetus brain electrical activity as well as muscular and cardiac signals on the mother's abdomen surface can be deployed in the selection of the algorithmic parameters such as: sampling rates, sensitivity thresholds, frequency bands (bands division and limiting). The following methods can be considered as pre-processing or post-processing analysis: 1) Linear and nonlinear methods; 2) Short time Fourier transform; 3) Wavelet transform; 4) principal and independent component analysis; 5) Empirical mode decompensation and 6) fractional dimension.

A potential roadblock of the first aim is the access to a clear fetal EEG signal. This may result from problems with the sensitivity of the electrodes, filtering of background noises from maternal and fetal origin, maternal habitus, position and station of the fetal head, the presence of polyhydramnios, etc. Alternative approaches for these potential roadblocks can include testing of several types of electrodes at various locations on the abdomen with ultrasonic guidance regarding the fetus head site, until the necessary signal is identified, it is desired to include mothers that their fetus is in breech presentation that will ease the recording. Identification of the fetal head position by ultrasound prior to EEG sampling and selection of the position with the minimal distance between the EEG recorder 114 and fetal head will assist in cases of maternal obesity.

It is known that the fetal brain develops with advancing gestation, especially during the second half of pregnancy. Women with low-risk pregnancies are followed prospectively along gestation and have fetal EEG reading at 6 week interval (8-14, 16-22, 22-28, 28-, 14, 34-37, >37 weeks of gestation).

At the scheduled prenatal visits, each patient has a 3D-minute noninvasive fetal EEG recording. The recording is captured and subsequently analyzed, and the pattern of EEG recorded is processed by the analytical team and will be reported based on the approach previously employed for neonatal EEG. The progressive collection of EEG recording with advancing gestation can be used in order to identify the assumed monotonically increase in EEG magnitude and use that information to create a more robust filtering and machine learning techniques that exploit that fact. An exemplary progression of EEG recordings throughout gestation (18 weeks, 22 weeks, 25 weeks, and 39 weeks) is shown in FIG. 6.

The comparative analysis of the EEG signal is performed in two dimensions, one longitudinally for each individual fetus and a second among the different fetuses in order to detect stereotypical patterns that can be used to enhance the algorithms and filtering methodology.

Thus, unsupervised learning techniques are use to extract fetus EEG, exploiting the above assumptions about the monotonic increase of EEG signal over time as well as the still typical features extracted from the comparative analysis of a cohort of different fetuses.

In an exemplary embodiment, a method of non-intrusively measuring brainwave activity in a fetus according to the present invention comprises the steps of: (a) directing at least a first monitoring sensor to a pregnant woman's body; (b) measuring a first set of signals received by the at least first monitoring sensor, which can be performed in a synchronous manner; (c) filtering non-fetal brainwave signals from the first set of signals, resulting in fetal brainwave activity signals; and (d) determining fetal brainwave activity from the fetal brainwave activity signals.

Prior to step (a), a clinician determines an approximate location of the head of the fetus and directing the at least first monitoring sensor, such as an electrode 116, proximate to the head. Step (a) can be performed by placing electrode on the abdomen of the mother. Alternatively, other methods, such as video recording the mother's abdomen and recording movements of the mother, EMG, respiratory, magnetic coils, galvanic sensors, and other known or yet unknown electrophysiological sensors and situation context monitors.

Steps (a) can be repeated a plurality of times; and a trend of fetal functional brain development can be determined with advancing gestation. Alternatively, or in addition, steps (a)-(d) can be repeated on a plurality of women and trends of the plurality of fetal functional brain developments can be determined. Any subsequent neonatal and childhood neurodevelopmental disorders can then be correlated with the trends. This correlation can be performed at least three years after birth of the fetus.

After the neonate is born, the process is repeated by directing at least a second monitoring sensor, such as a second electrode 116, to the neonate's head; measuring a second set of signals received by the monitoring sensor; filtering non-neonatal brainwave signals from the second set of signals, resulting in neonatal brainwave activity signals; and determining neonatal brainwave activity from the neonatal brainwave activity signals. The pre-birth step of determining the fetal brainwave activity is performed approximately one hour before delivery of the fetus and the step of determining the neonatal is performed within eight hours of the delivery of the fetus.

In an alternative embodiment, a method of non-intrusively measuring brainwave activity in a fetus can include the steps of: (a) receiving data from a first set of sensors; (b) filtering non-fetal brainwave signals from the data, resulting in fetal brainwave activity signals; and (c) determining fetal brainwave activity from the fetal brainwave activity signals. Steps (a)-(c) immediately above can be repeated a plurality of times and a trend of fetal functional brain development and well-being based on the fetal brainwave activity can be determined.

In another alternative embodiment, a method of non-intrusively measuring brainwave activity in a fetus includes the steps of: (a) directing at least a first monitoring sensor to a pregnant woman's body; (b) measuring a first set of signals received by the at least first monitoring sensor; (c) repeating steps (a) and (b) a plurality of times; and (d) using an independent method to identify fetal brainwave signals in the first set of signals.

Regarding step (d), several independent methods can be used to identify and isolate the fetal brainwave signals from other signals that can be measured and recorded. Exemplary non-fetal brainwave activity can include at least one of respiratory activity, cardiac activity, digestion processes, and muscle movement, among other signals. When these signals are identified, these signals can be eliminated from the analysis of the measured and recorded signals, leaving the fetal brainwave signals to be analyzed.

An exemplary first method can include identifying a subset of signals in the first set of signals, wherein the subset of signals increases in strength and evolves monotonically throughout step (c). Specifically, the strength (average power) of the extracted signal is increasing with each increasing week of pregnancy.

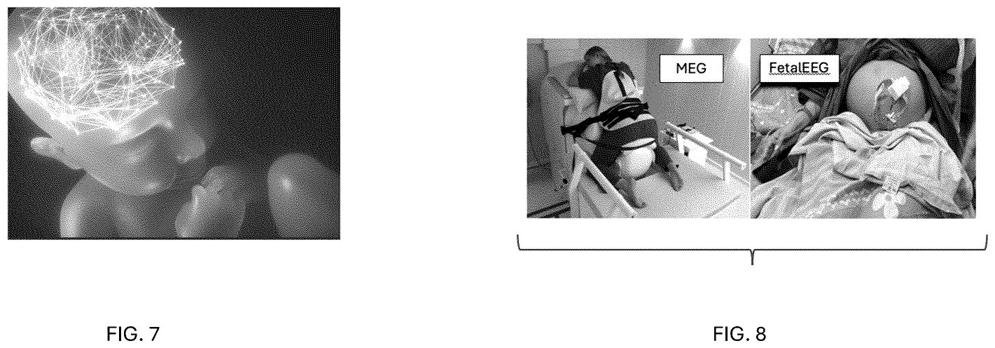

An exemplary second method can include comparing features biomarkers to the first set of signals, as shown in FIG. 7. Using the features biomarkers specific features of neonatal EEG are examined in term fetuses, including brain activity and recordings.

An exemplary third method can include using a magnetic coil to record data and comparing the data to the first set of signals. The magnetic coil can include a magnetoenchephalograph (MEG) machine, as shown in FIG. 8, or some other magnetic coil. Data obtained from the MEG is then compared to data obtained from the fetal EEG.

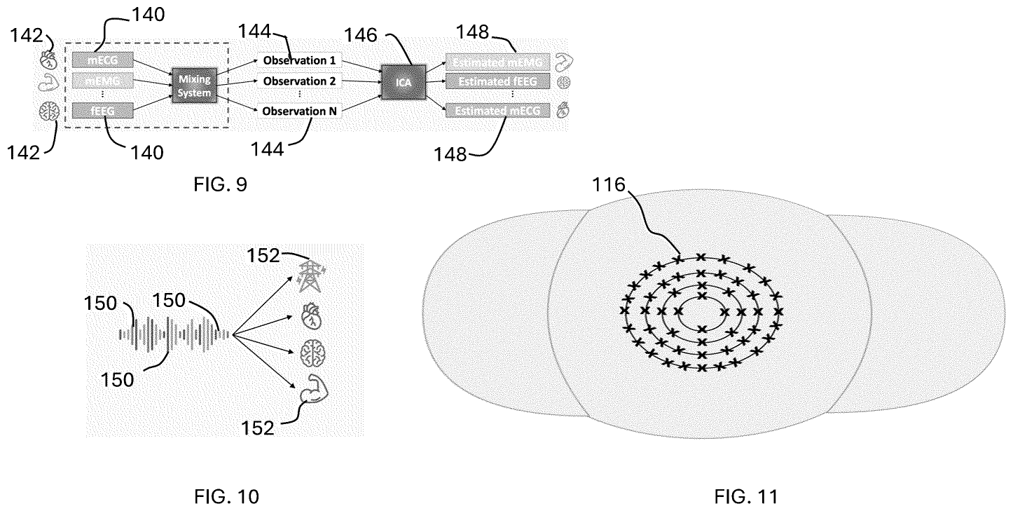

An exemplary fourth method can include using Blind Source Separation (BSS), shown in FIGS. 9 and 10, to estimate sources of the first set of signals by treating each disturbance as a source; suppressing disturbance sources; and estimating fetal brainwave activity. Referring specifically to FIG. 9, a plurality of signals 140 from different sources 142 are mixed together and, through observations 144 and Independent Computer Analysis (ICA) 146, estimated results 148 attributable to each source 142 are determined. Alternatively, as shown in FIG. 10, different sources 150 can have different amplitudes and possibly also different frequencies, which are mapped to separate functions 152, from which fetal neurological signals can be mapped BSS can be used to compare to recorded values to validate results.

An exemplary fifth method can include placing a plurality of sensors on the pregnant woman's body in concentric contoured patterns, such as ellipses, such that the contours having different radii. An exemplary pattern of sensors includes electrodes 116 placed on the body as shown in FIG. 11.

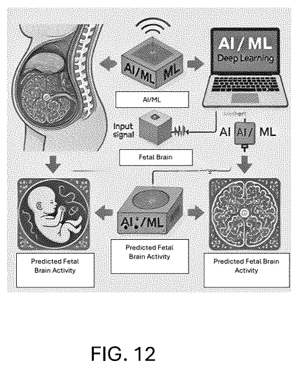

An exemplary seventh method can include using artificial intelligence and machine and deep learning methods, shown in an exemplary embodiment in FIG. 12, where any box with artificial intelligence/machine learning is configured so that the input is signal from mother, and the output is predicted fetal brain signal.

An exemplary seventh method can include repeating steps (a)-(c) above on a plurality of women; labeling the fetal brainwave signals, forming labeled datasets; using the labeled datasets to train machine learning and deep learning models; and using the models as a baseline to identify signals received from a new pregnant woman.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A method of non-intrusively measuring brainwave activity in a fetus, the method comprising the steps of:
(a) directing at least a first monitoring sensor to a pregnant woman's body;
(b) measuring a first set of signals received by the at least first monitoring sensor;
(c) filtering non-fetal brainwave signals from the first set of signals, resulting in fetal brainwave activity signals; and
(d) determining fetal brainwave activity from the fetal brainwave activity signals.

2. The method according to claim 1, further comprising, prior to step (a), determining an approximate location of the head of the fetus and directing the at least first monitoring sensor proximate to the head.

3. The method according to claim 1, further comprising performing steps (a)-(d) prior to a Caesarian Section birth of the fetus, resulting in a neonate, and then performing the following steps on the neonate:
(e) directing at least a second monitoring sensor to the neonate's head;
(f) measuring a second set of signals received by the monitoring sensor;
(g) filtering non-neonatal brainwave signals from the second set of signals, resulting in neonatal brainwave activity signals; and
(h) determining neonatal brainwave activity from the neonatal brainwave activity signals.

4. The method according to claim 3, wherein step (d) is performed approximately one hour before delivery of the fetus and step (f) is performed within eight hours of the delivery of the fetus.

5. The method according to claim 1, further comprising the steps of:
(e) repeating steps (a)-(d) a plurality of times; and
(f) determining a trend of fetal functional brain development with advancing gestation.

6. The method according to claim 1, further comprising:
(e) repeating steps (a)-(d) on a plurality of women;
(f) determining trends of the plurality of fetal functional brain developments; and
(g) correlating any subsequent neonatal and childhood neurodevelopmental disorders.

7. The method according to claim 1, wherein step (g) is performed at least three years after birth of the fetus.

8. The method according to claim 1, wherein the non-fetal brainwave activity comprises at least one of respiratory activity, cardiac activity, digestion processes, and muscle movement.

9. The method according to claim 1, wherein step (c) comprises maximizing a signal-to-noise ratio of the first set of signals.

10. The method according to claim 1, wherein step (a) comprises applying a plurality of the first monitoring sensors in a concentric contour pattern.

11. The method according to claim 10, wherein step (b) comprises collecting the first set of signals in a synchronized manner.

12. The method according to claim 1, further comprising, after step (d), comparing results from step (d) to an alternative measurement method.

13. A method of non-intrusively measuring brainwave activity in a fetus, the method comprising the steps of:
(a) receiving data from a first set of sensors;
(b) filtering non-fetal brainwave signals from the data, resulting in fetal brainwave activity signals; and
(c) determining fetal brainwave activity from the fetal brainwave activity signals.
(d) repeating steps (a)-(c) a plurality of times; and
(e) determining a trend of fetal functional brain development and well-being based on the fetal brainwave activity.

14. A method of non-intrusively measuring brainwave activity in a fetus, the method comprising the steps of:
(a) directing at least a first monitoring sensor to a pregnant woman's body;
(b) measuring a first set of signals received by the at least first monitoring sensor;
(c) repeating steps (a) and (b) a plurality of times; and
(d) using an independent method to identify fetal brainwave signals in the first set of signals.

15. The method according to claim 14, wherein the independent method comprises identifying a subset of signals in the first set of signals, wherein the subset of signals increases in strength and evolves monotonically throughout step (c).

16. The method according to claim 14, wherein the independent method comprises comparing features biomarkers to the first set of signals.

17. The method according to claim 14, wherein the independent method comprises using a magnetic coil to record data and comparing the data to the first set of signals.

18. The method according to claim 14, wherein the independent method comprises the steps of:
(a) using blind source separation to estimate sources of the first set of signals;
(b) treating each disturbance as a source;
(c) suppressing disturbance sources; and
(d) estimating fetal brainwave activity.

19. The method according to claim 14, wherein step (a) comprises placing a plurality of sensors on the pregnant woman's body in concentric contours, the contours having different radii.

20. The method according to claim 14, wherein the independent method comprises the steps of:
(d1) repeating steps (a)-(c) on a plurality of women;
(d2) labeling the fetal brainwave signals, forming labeled datasets;
(d3) using the labeled datasets to train machine learning and deep learning models; and
(d4) using the models as a baseline to identify signals received from a new pregnant woman.
